(19) United States
(12) Patent Application Publication
(10) Pub. No.: US 2026/0345842 A1
(43) Pub. Date: Nov. 5, 2026

(54) ELECTRON BEAM RADIATION FOR THE STERILIZATION OF MEDIA PRIOR TO INOCULATION WITH FUNGI AND RELATED METHODS

(71) Applicant: SYLVAN, INC., Kittanning, PA (US)

(72) Inventor: Mark WACH, Allison Park, PA (US)

(21) Appl. No.: 19/101,785

(22) PCT Filed: Aug. 18, 2023
(86) PCT No.: PCT/US2023/072440

§ 371 Date: Feb. 6, 2025

Related U.S. Application Data
(60) Provisional application No. 63/399,425, filed on Aug. 19, 2022.

(51) Int. Cl. A61L 2/087; A01G 18/20; A01G 18/30; A01G 18/50; A61L 2/082; A61L 103/05

(52) U.S. Cl. CPC A61L 2/087; A01G 18/20; A01G 18/30; A01G 18/50; A61L 2/082; A61L 2103/05

(57) ABSTRACT

The present invention provides a continuous process for the sterilization of a substrate. The process comprising a step of preparing a substrate mixture having a moisture content. A further step includes loading a carrier with the substrate mixture, while the next step includes exposing the substrate mixture to electron beam ionizing radiation in an electron beam bunker housing to create a sterilized substrate having the same moisture content as the substrate mixture. The final step includes unloading the sterilized substrate from the carrier such that the carrier can be reused in the continuous process.

### CROSS REFERENCE TO RELATED APPLICATIONS

This application claims the benefit of U.S. Provisional Patent Application No. 63/399,425, filed Aug. 19, 2022, the subject matter of which is incorporated herein by reference.

### FIELD OF INVENTION

The present invention generally relates to the use of ionizing irradiation, including electron beam radiation or X-ray radiation, for the sterilization of substrates or other media prior to inoculation with various fungi. The present invention further relates to uniformly blending, uniformly hydrating, and sterilizing via ionizing (i.e., electron beam or X-ray) radiation of various media or substrates without heat or steam during sterilization, in a continuous process prior to inoculation with various fungi. The method of the present invention provides for a more accurate moisture content and better mimics the natural particulates comprising the substrates or media, while creating a much more efficient process for the cultivation of mushrooms or other fungi.

### BACKGROUND OF THE INVENTION

It is generally understood that mushroom and other fungal cultures, much like those of other microorganisms, are prepared, maintained, propagated, and stored on sterile media using microbiological laboratory methods. Sterile tools and aseptic techniques are used within clean rooms or sterile transfer hoods or carriers to manipulate cells of the pure cultures for various purposes including clonal propagation, the development of new strains, and generally for production or farming of such cultures. There are a variety of diverse techniques that can be used, including spore germinations on sterile growth media and controlled matings on sterile growth media. Commercial culture inocula and production materials including mushroom spawn and casing inoculum. Synthetic “logs” are also prepared using large-scale microbiological production methods, for example by aseptically introducing inoculum of a pure culture of a fungal strain into from one to 14,000 liters of sterilized growth media under aseptic conditions. Often the inoculated cultures are provided to the end user as pure cultures on sterilized growth media contained within aseptic packaging.

Mushrooms or other fungal products are then cultivated commercially within purpose-built structures on dedicated facilities. While there are many variations on methods, the following description is typical. Substrates prepared from lignocellulosic materials such as straw, sawdust, or wood chips, augmented with a nitrogenous material, is finished, and pasteurized or sterilized within a suitable facility. Mushroom spawn or other suitable inoculum, which comprises a sterilized friable ‘carrier substrate’ or liquid medium in which a pure culture of one or more fungal strains have been aseptically incorporated via inoculum and then propagated, is mixed with the prepared substrate, and is incubated for approximately 10 to about 90 days at a controlled temperature, during which time the mycelium of the mushroom culture colonizes the entire mass of substrate and begins to digest it. For some mushroom forming fungi, including Agaricus bisporus, a non-nutritive ‘casing layer’ of material such as peat may then be placed over the substrate to a depth of from about 1.5 to about 2 inches. Additional ‘casing inoculum’ incorporating the same mushroom culture may be incorporated into the casing layer to accelerate the formation and harvesting of fungal biomass such as mushrooms and improve uniformity of the distribution of mycelium in and on the casing surface. Environmental conditions, including temperature and humidity, in the production facility are then carefully managed to promote and control the transition of the culture from vegetative to reproductive growth at the casing/air interface. For Agaricus bisporus, in about 13 to 18 days after casing, mushrooms will have developed to the correct stage for harvest and sale. A flush of mushrooms comprising the original culture will be picked over a 3-to-4-day period. Additional flushes of mushrooms appear at about weekly intervals. Commercially, two or three flushes of mushrooms are produced and harvested before the compost is removed and replaced in the cropping facility.

In the cultivation of mushroom spawn or other fungal based products, sterilization of the substrate prior to inoculation with the appropriate fungal culture is necessary in order to avoid the growth of other bacteria and molds which would inhibit the growth of the desired organism. In conventional methods of cultivating such materials, the sterilization of the substrate is labor intensive and performed by “batch” processing, meaning only a certain amount can be sterilized at any one time, depending upon the capacity of the heating apparatus. That is, the sterilization process is typically undertaken at high temperatures well above 35 °C (more likely above 50 °C, and even more likely, in the 100-125 °C range) within a kiln or autoclave, or some other heating apparatus for a significant period of time, namely at least 12 to 24 hours, which lowers the moisture content of the substrate and may alter the texture and nutrient value of the substrate, which is an unwanted characterization of conventional methods. Another drawback of having to sterilize substrates in high heat is that such a process has to be undertaken as a batch process, as opposed to a more efficient continuous process, due to the heated substrate needing time to cool. Therefore, there is a need in the art for a method of sterilizing a substrate prior to inoculation that avoids the pitfalls of previous methods such as the unnecessary lowering of the moisture content of the substrate. The need for a more efficient process for the cultivation of mushrooms or other fungi also exists.

### SUMMARY OF THE INVENTION

At least one aspect of the present invention provides a continuous process for the sterilization of a substrate, the process comprising the steps of: preparing a substrate mixture having a moisture content; loading a carrier with the substrate mixture; exposing the substrate mixture to electron beam or X-ray ionizing radiation in an electron beam or X ray bunker housing to create a sterilized substrate having the same moisture content as the substrate mixture; and unloading the sterilized substrate from the carrier such that the carrier can be reused in the continuous process. In one or more embodiments, the process steps may be sequential.

Another embodiment of the present invention provides a continuous process as in the embodiment above, further comprising a step of packaging the sterilized substrate into a package for transport. In some embodiments, this would be a step after the step of unloading.

Another embodiment of the present invention provides a continuous process as in the embodiment above, further comprising a step of inoculating the sterilized substrate with an inoculum to create an inoculated substrate. In some embodiments, this would be a step after the step of unloading.

Another embodiment of the present invention provides a continuous process as in the embodiment above after the step of inoculating, further comprising a step of packaging the inoculated substrate into a package for transport.

Another embodiment of the present invention provides a continuous process as in the embodiment above, wherein the inoculum is an organism selected from, but not limited to, the group consisting of Agaricus spp. Morchella spp. Trichoderma spp., Beauvaria spp., Lntinula spp., Ganoderma spp, or Metahrizium spp. It will be appreciated that the present invention is not necessarily limited to these particular spore forming organisms. These just happen to presently be the most commercially acceptable organisms.

Another aspect of the present invention provides a continuous process as in any embodiments above, wherein during the step of unloading the sterilized substrate, the carrier is taken to a clean room directly adjacent the electron beam or X-ray bunker housing after being exposed to the electron beam or X-ray ionizing radiation, and the step of unloading takes place within the clean room.

Another embodiment of the present invention provides a continuous process as in any embodiments above, wherein during the step of unloading the sterilized substrate, the carrier is transported to a clean room that is not directly adjacent the electron beam or X-ray bunker housing after being exposed to the electron bean ionizing radiation.

Another embodiment of the present invention provides a continuous process as in any embodiments above, wherein the substrate mixture includes nutrient particles and optionally added water.

Another embodiment of the present invention provides a continuous process as in any embodiments above, wherein the nutrient particles are selected from the group consisting of sawdust, fermented sawdust, millet, sorghum, grain, agricultural waste, oilseed products, cereal, formulated compost, fermented compost, peat moss, gypsum, chalk, vermiculate, perlite, clay, wastepaper materials, and combinations thereof.

Another embodiment of the present invention provides a continuous process as in any embodiment wherein the moisture content of the substrate mixture is between 30% and 70%.

Another embodiment of the present invention provides a continuous process as in any embodiment, wherein the step of exposing includes subjecting the substrate mixture to an ionizing radiation dosage of between about 5 kGy to about 40 kGy.

Another embodiment of the present invention provides a continuous process as in any embodiment, wherein the step of exposing takes place at a temperature of from between about 10 °C and about 35 °C.

It will be appreciated that these and other aspects of the present invention provide advantages in the sterilization of media and other substrates not previously obtained by heating the substrates. The amount of time for conducting this continuous process is seconds to minutes (up to one hour or less, but often only 1 minute to 10 minutes) compared to the batch processing of the prior art, wherein the bulk substrates must be heated and cooled over a significant period of time (12-24 hours or more), and wherein the substrates lose at least some of their moisture content.

It will also be appreciated that, while electron beam and/or X-ray radiation is known in the art of sterilization, it has never been conducted within a continuous process for the sterilization of substrate media for the production of mushrooms or other fungi, prior to inoculation. In fact, if used previously, electron beam radiation has only been used on final products, which in the case of fungi, would also mean killing the inoculated materials sought to be grown on the substrates set forth in this description. In this invention, electron beam and X-ray radiation is being used to sterilize media so that something else can grow, not be further sterilized.

### BRIEF DESCRIPTION OF THE DRAWINGS

For a more complete understanding of the features and advantages of the present invention, reference is now made to the detailed description of the invention along with the accompanying figures in which:

FIG. 1 illustrates a simplified representation of a first embodiment of a continuous process for the sterilization of a substrate;

FIG. 2 illustrates a simplified representation of a second embodiment of a continuous process for the sterilization of a substrate;

FIG. 3 illustrates a simplified representation of a third embodiment of a continuous process for the sterilization of a substrate;

FIG. 4 illustrates a simplified representation of a fourth embodiment of a continuous process for the sterilization of a substrate.

### DETAILED DESCRIPTION OF ILLUSTRATIVE EMBODIMENTS

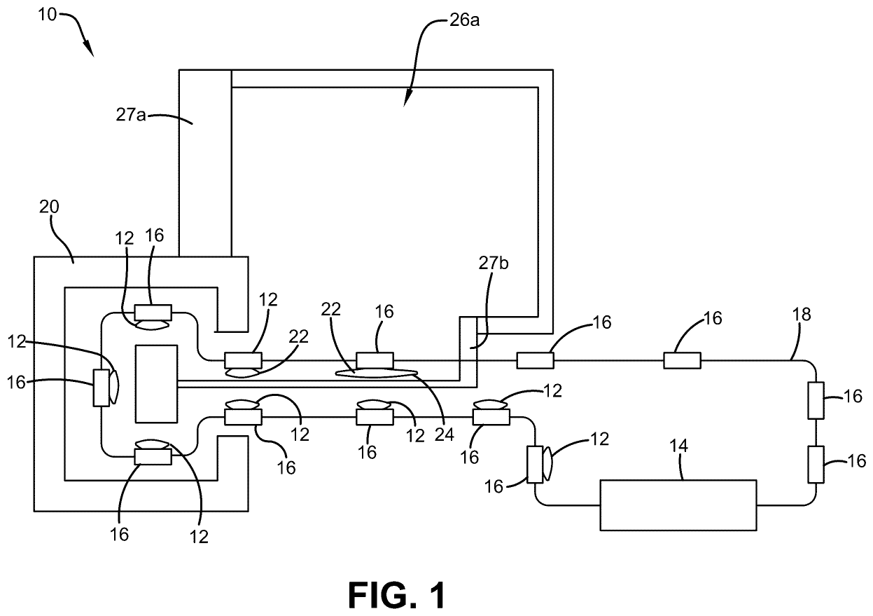

The present invention provides a continuous process that utilizes electron beam radiation for the sterilization of substrates or other media prior to inoculation with various fungi. FIG. 1 shows a simplified representation of a continuous process 10 for the sterilization of a substrate mixture 12. In the first step of the process 10, the substrate mixture 12 is prepared in a mixer 14. The substrate mixture 12 will include nutrient particles and optionally added water to provide the substrate mixture 12 with a moisture content. Once the substrate mixture 12 is prepared, the next step is to load the substrate mixture 12 onto a carrier 16. The carrier 16 is then taken along a conveyer 18 into an electron beam bunker housing 20 (or alternatively, an X-ray bunker housing) to expose the substrate mixture 12 to electron beam ionizing radiation to create a sterilized substrate 22. It is to be understood that X-ray irradiation may be substituted for electron beam irradiation throughout this application, and that an “X-ray bunker housing” can be substituted for “electron beam bunker housing” as used in this application. In one or more embodiments, the carrier 16 is a covered carrier which utilizes a lid or similar object to contain the substrate mixture 12 within the carrier 16. In other embodiments, the carrier 16 is an uncovered carrier which leave the substrate mixture 12 open to the environment.

It is important to note that the process 10 of the present invention is a continuous process, which means that the carriers 16 can continuously travel around the conveyor 18 and be used multiple times with the process 10 of the present invention.

It is important to note that the continuous process 10 of the present invention also does not heat the substrate mixture 12 in order to sterilize the substrate mixture. This is an advantage over previous methods of sterilizing substrate mixtures, as there is no loss of moisture within the substrate mixture 12 as it is sterilized.

It is important to note that in the continuous process 10 of the present invention, the sterilized substrate 22, having been exposed to electron beam ionizing radiation in the electron beam bunker housing 20, will have the same moisture content as the substrate mixture 12 prior to the substrate mixture 12 being exposed to the electron beam ionizing radiation in the electron bean bunker housing 20.

The next step in the continuous process 10 is unloading of the sterilized substrate 22 from the carrier 16 such that the carrier 16 can be taken back along the conveyer 18 and be reused in the continuous process 10. In some embodiments, the act of unloading the sterilized substrate 22 from the carrier 16 is done by a pneumatic carrier flipper 24, such as shown in FIG. 1. FIG. 1 also shows that the unloading of the sterilized substrate 22 from the carrier 16 is done in a clean room 26a that is directly adjacent the electron beam bunker housing 20. Directly unloading in a clean room 26a will allow for additional steps, which will be discussed later, to occur directly after the sterilized substrate 22 is unloaded from the carrier 16. As shown in FIG. 1, the clean room 26a include HEPA walls 27a and 27b. The HEPA walls 27a and 27b supply a clean, filtered, and laminar flow of air across the enclosed space allowing workers to operate in the clean room while minimizing the likelihood of contaminating any exposed sterilized substrate 22.

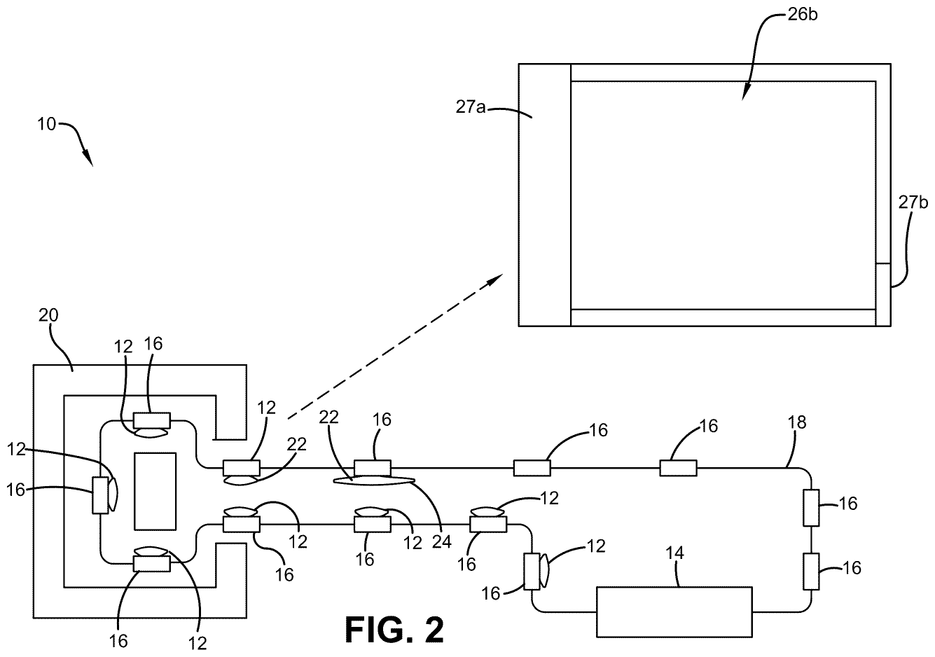

In another embodiment, as shown in FIG. 2, the act of unloading the sterilized substrate 22 from the carrier 16 is not done in a clean room. In this embodiment, the substrate is covered or placed in a sealed carrier, and sterilized, Once unloaded, the sterilized substrate 22 is transported to a clean room 26b that is not directly adjacent the electron beam bunker housing 20. The differences between the embodiment of FIG. 1 and the embodiment of FIG. 2 will be based on design parameters of the facility wherein the process 10 is taking place. If the facility has the ability to set up a clean room 26a that is directly adjacent the electron beam bunker housing 20, then they have the option to do so. If the facility does not have room to place a clean room 26b that is directly adjacent to the electron beam bunker housing 20, then they have the option to place the clean room 26b not directly adjacent the electron beam bunker housing 20. The location of the clean room 26a/26b does not change the scope of the present invention.

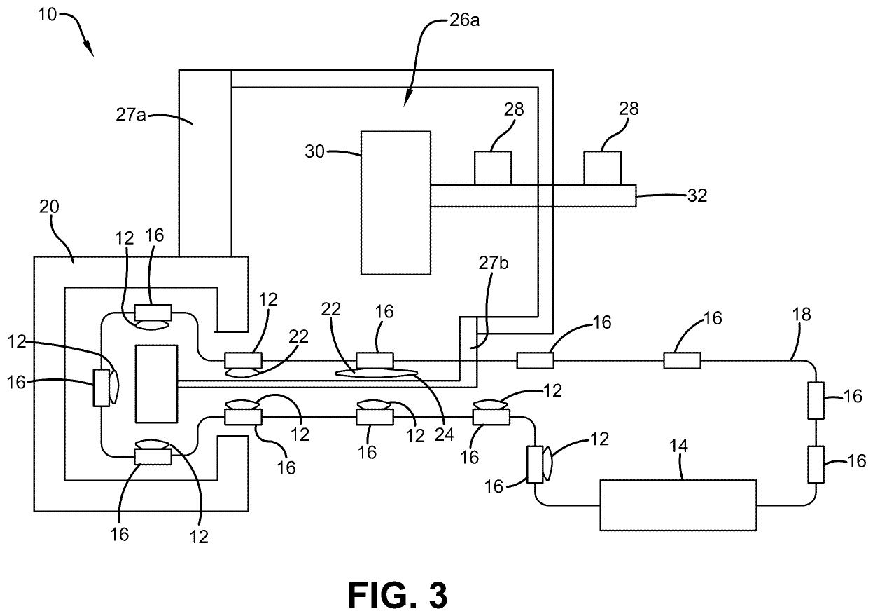

In another embodiment, as shown in FIG. 3, the continuous process 10 includes an additional step, which takes place after the step of unloading, of packaging the sterilized substrate 22 into a package 28 for transport. The step of packaging is shown in FIG. 3 as being done by a bagger or auto bagger 30. Once the sterilized substrate 22 has been packaged into a package 28, it can be transported to a different facility for inoculation. As shown in FIG. 3, the packages 28 are initially taken from the bagger or auto bagger along a conveyer 32. Although FIG. 3 shows the bagger or auto bagger 30 within the confines of the clean room 26a, it is contemplated that the bagger or auto bagger 30 can be located outside the confines of a clean room 26a.

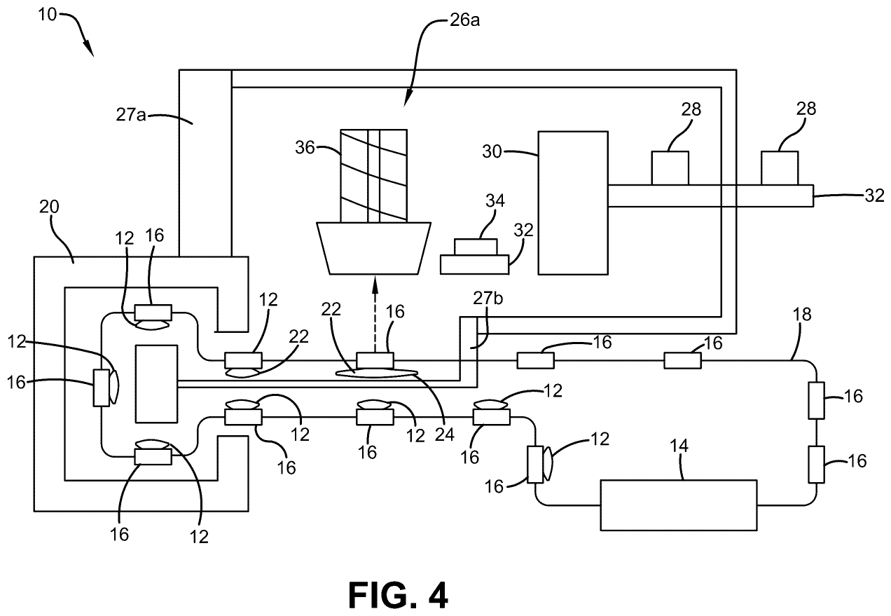

In another embodiment, as shown in FIG. 4, the continuous process 10 includes an additional step, which takes place after the step of unloading, of inoculating the sterilized substrate 22 with inoculum to create an inoculated substrate 34. The step of inoculation is shown in FIG. 4 as being done by an inoculation mixing screw 36, but the present invention is not limited to such an inoculation device. It is important to note that the inoculation mixing screw 36 will need to be located within a clean room 26a. FIG. 2 also shows an inoculation mixing screw 36 located within clean room 26b.

FIG. 4 also shows an additional step, which would take place after the step of inoculation, of packaging the inoculated substrate 34 into a package 28 for transport. The step of packaging is shown in FIG. 4 as being done by a bagger or auto bagger 30. Once the inoculated substrate 34 has been packaged into a package 28, it can be transported to a customer to harvest what is grown by the inoculated substrate 34. Although FIG. 4 shows the bagger or auto bagger 30 within the confines of the clean room 26a, it is contemplated that the bagger or auto bagger 30 can be located outside the confines of a clean room 26a.

Throughout the continuous process 10, as shown in any of the embodiments in FIGS. 1-4, the substrate mixture 12 includes nutrient particles and optionally added water. The nutrient particles are selected from the group consisting of sawdust, fermented sawdust, millet, sorghum, grain, oilseed products, agricultural waste, cereal, formulated compost, fermented compost, peat moss, gypsum, chalk, vermiculate, perlite, clay, wastepaper materials, and combinations thereof. The present invention contemplates that the moisture content of the substrate mixture 12 should be between 30% and 70%. Dependent upon the moisture content of the nutrient particles by themselves, and the desired moisture content of the substrate mixture 12, water may be added to the substrate mixture.

Throughout the continuous process 10, as shown in any of the embodiments in FIGS. 1-4, during the step of exposing the substrate mixture 12 to electron beam ionizing radiation in an electron beam bunker housing 20 to create a sterilized substrate 22 having the same moisture content as the substrate mixture 12, the substrate mixture 12 is exposed to an ionizing radiation dosage of between about 5 kGy to about 40 kGy. The dosage amount of ionizing radiation is determined based on the thickness of the substrate mixture 12 being treated, with an effective penetration of the ionizing radiation being dependent on the thickness of the substrate mixture 12. Additionally, the step of exposing takes place at a temperature of from between about 10 °C and about 35 °C.

Throughout the continuous process 10, as shown in any of the embodiments in FIGS. 1-4, it should be noted that the electron beam ionizing radiation applied to the substrate mixture 12 within the electron beam bunker housing 20 can only penetrate down to 9 cm within the substrate mixture 12. Therefore, if the substrate mixture 12 is thicker than 9 cm, it is contemplated that the substrate mixture can travel through the electron beam bunker housing 20 more than one time, or through multiple beams (i.e., above, and below). In some embodiments, the substrate mixture 12 travels through the electron beam bunker housing 20 a first time, the substrate mixture 12 is then rotated 180 degrees and it then travels through the electron beam bunker housing 20 a second time. In some embodiments, the substrate mixture 12 travels through the electron beam bunker housing 20 a first time, the substrate mixture 12 is then rotated 90 degrees and it then travels through the electron beam bunker housing 20 a second time, the substrate mixture 12 is then rotated an additional 90 degrees and it then travels through the electron beam bunker housing 20 a third time, and then the substrate mixture 12 is then rotated an additional 90 degrees and it then travels through the electron beam bunker housing 20 a fourth and final time.

In the embodiment of the continuous process 10 of the present invention as shown in FIG. 4, the inoculum used to inoculate the sterilized substrate 22 to create an inoculated substrate 32 is a spore forming organism selected from the group consisting of Agaricus spp., Morchella spp., Trichoderma spp., Beauvaria spp., Lentinula spp., Ganoderma spp., or Metahrizium spp. This list of inoculum is only a limited list of potential inoculum that can be utilized in the continuous process 10 of the present invention, other different types of fungi not listed here are also contemplated for use in the continuous process 10 of the present invention.

In light of the foregoing, it should be appreciated that the present invention significantly advances the art by providing a continuous process for the sterilization of a substrate that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

In a first Example, cooked millet, milo (sorghum), and rye grains having a moisture content between 43% and 46% were sampled to determine their initial biological load (shown in Table 1 below). Then 2.5 pound sampled were placed in suitable sealed plastic containers prior to exposure to varying levels of electron beam ionizing radiation of between 5.96 kGy and 15.8 kGy by adjusting the speed at which the substrate mixtures were passed through the exposure field created in the electron beam bunker housing.

TABLE 1
Biological Load in Untreated Nutrient Particles

| | Bacteria | Fungus |
|---|---|---|
| | Concentration | Concentration |
| Sample Description | (CFU₁/g) | (CFU/g) |
| Untreated millet | 2.00E+02 | BMDL₂ |
| Untreated rye grains | BMDL | BMDL |
| Untreated milo | 2.10E+03 | BMDL |

₁Colony Forming Units
₂Below Minimum Detection Levels which equates to <200 CFU/g

Following exposure, the contents of each container was aseptically sampled to determine the degree of sterility achieved (results shown in Tables 2, 3 and 4 below). The remainder of the treated material was inoculated with a suitable source of commercial Agaricus bisporus inoculum and the degree of colonization was observed over the next two to three weeks and compared to a commercial control. The degree of colonization was observed on three different media: potato dextrose agar, tryptic soy agar, and tryptic soy agar. The potato dextrose agar is a microbiological growth medium used for fungi, while the tryptic soy agar is a microbiological growth medium used for bacteria. The tryptic soy both is a common growth medium used for low level detection of bacteria. Enrichment of the broth includes a 5-day incubation of the broth boxes, followed by a sterility determination performed by streaking the broth on tryptic soy agar plates.

TABLE 2
Electron beam ionizing radiation treated Millet Sterility Check

| | | Actual | | | |
|---|---|---|---|---|---|
| | Conveyer | Dose of | | | |
| Product | Speed | radiation | PDA₃ | TSA₄ | TSB₅ |
| Description | (FPM) | (kGy) | Plate | Plate | Plate |
| Millet | 5.41 | 16.4 | No | No | No |
| | | | Contami- | Contami- | Contami- |
| | | | nation | nation | nation |
| Millet | 6.17 | 13.8 | No | No | No |
| | | | Contami- | Contami- | Contami- |
| | | | nation | nation | nation |
| Millet | 7.17 | 11.2 | No | No | No |
| | | | Contami- | Contami- | Contami- |
| | | | nation | nation | nation |
| Millet | 8.56 | 9.2 | No | No | No |
| | | | Contami- | Contami- | Contami- |
| | | | nation | nation | nation |
| Millet | 10.63 | 8.2 | No | No | No |
| | | | Contami- | Contami- | Contami- |
| | | | nation | nation | nation |
| Millet | 14.00 | 6.2 | Contami- | No | No |
| | | | nation | Contami- | Contami- |
| | | | | nation | nation |

₃Potato Dextrose Agar
₄Tryptic Soy Agar
₅Tryptic Soy Broth

TABLE 3
Electron beam ionizing radiation treated Milo Sterility Check

| | | Actual | | | |
|---|---|---|---|---|---|
| | Conveyer | Dose of | | | |
| Product | Speed | radiation | PDA | TSA | TSB |
| Description | (FPM) | (kGy) | Plate | Plate | Plate |
| Milo | 5.41 | 16.4 | No | No | No |
| | | | Contami- | Contami- | Contami- |
| | | | nation | nation | nation |
| Milo | 6.17 | 13.8 | No | No | Contami- |
| | | | Contami- | Contami- | nation |
| | | | nation | nation | |
| Milo | 7.17 | 11.2 | No | No | No |
| | | | Contami- | Contami- | Contami- |
| | | | nation | nation | nation |
| Milo | 8.56 | 9.2 | No | Contami- | No |
| | | | Contami- | nation | Contami- |
| | | | nation | | nation |
| Milo | 10.63 | 8.0 | No | No | No |
| | | | Contami- | Contami- | Contami- |
| | | | nation | nation | nation |
| Milo | 14.00 | 5.8 | No | Contami- | No |
| | | | Contami- | nation | Contami- |
| | | | nation | | nation |

TABLE 4
Electron beam ionizing radiation treated Rye Grain Sterility Check

| | | Actual | | | |
|---|---|---|---|---|---|
| | Conveyer | Dose of | | | |
| Product | Speed | radiation | PDA | TSA | TSB |
| Description | (FPM) | (kGy) | Plate | Plate | Plate |
| Rye Grain | 5.41 | 15.8 | No | No | Contami- |
| | | | Contami- | Contami- | nation |
| | | | nation | nation | |
| Rye Grain | 6.17 | 13.6 | No | No | No |
| | | | Contami- | Contami- | Contami- |
| | | | nation | nation | nation |
| Rye Grain | 7.17 | 11.5 | No | No | No |
| | | | Contami- | Contami- | Contami- |
| | | | nation | nation | nation |
| Rye Grain | 8.56 | 9.4 | No | No | No |
| | | | Contami- | Contami- | Contami- |
| | | | nation | nation | nation |
| Rye Grain | 10.63 | 7.7 | No | No | No |
| | | | Contami- | Contami- | Contami- |
| | | | nation | nation | nation |
| Rye Grain | 14.00 | 5.9 | Contami- | No | No |
| | | | nation | Contami- | Contami- |
| | | | | nation | nation |

In a second example, a mixed particulate substrate containing an inert solid support (vermiculate, perlite, Biodac mixture), and a representative nutrient mixture of corn meal, feather meal, wheat bran, soybean meal, yeast, chalk, gypsum, lime, and composted wheat straw were combined and mixed with water to achieve a homogeneous substate mixture having a moisture content of between 43% and 46% and a pH suitable for growing fungal cells. The material was sampled to determine the initial biological load (Table 5). Then, 2.5-pound samples were placed in suitable sealed plastic containers prior to exposure to varying levels of electron beam ionizing radiation of between 5.9 kGy and 16.1 kGy by adjusting the speed at which the substrate mixture was passed through the exposure field created in the electron beam bunker housing.

TABLE 5
Biological Load of Untreated mixed particulate substrate

| Bacteria Concentration (CFU/g) | Fungus Concentration (CFU/g) |
|---|---|
| 1.34E+08 | BMDL |

Following exposure, the contents of each container was aseptically sampled to determine the degree of sterility achieved (Table 6). The remainder of the treated material was inoculated with a suitable source of commercial Agaricus bisporus inoculum and the degree of colonization was observed over the next two to three weeks and compared to a commercial control.

TABLE 6
Electron beam ionizing radiation treated mixed
particular substrate Sterility Check

| | | Actual | | | |
|---|---|---|---|---|---|
| | Conveyer | Dose of | | | |
| Product | Speed | radiation | PDA | TSA | TSB |
| Description | (FPM) | (kGy) | Plate | Plate | Plate |
| Mixed | 5.41 | 16.15 | No | Contami- | No |
| Particulate | | | Contami- | nation | Contami- |
| | | | nation | | nation |
| Mixed | 6.17 | 13.6 | No | Contami- | No |
| Particulate | | | Contami- | nation | Contami- |
| | | | nation | | nation |
| Mixed | 7.17 | 11.9 | Contami- | Contami- | Contami- |
| Particulate | | | nation | nation | nation |
| Mixed | 8.56 | 9.7 | Contami- | Contami- | Contami- |
| Particulate | | | nation | nation | nation |
| Mixed | 12.32 | 6.75 | Contami- | Contami- | Contami- |
| Particulate | | | nation | nation | nation |

In a third example, a mixed substrate containing sawdust, wheat bran and gypsum was mixed with water to achieve a homogenous substrate material having a moisture content of between 45% and 65% and a pH suitable for growing fungal cells. The material was sampled to determine the initial biological load (Table 7). Then, 2.5 Kg samples were placed in suitable sealed cylindrical plastic tubes and compacted to a density of 0.9 g/cm3 prior to exposure to a fixed level of ionizing radiation of 10 kGy at a speed of 4.92 FPM through the exposure field created in the electron beam bunker housing. The bags were rotated either 180 degrees and passed through the field a second time or passed through the field a total of four times rotating the cylinder in 90-degree increments.

TABLE 7
Biological Load of Untreated mixed sawdust substrate

| Bacteria Concentration (CFU/g) | Fungus Concentration (CFU/g) |
|---|---|
| 7.5E+04 | 5.6E+06 |

Following exposure, the sterilized substrate in each bag were aseptically sampled to determine the degree of sterility achieved (Table 8). The remainder of the sterilized substrate material was inoculated with a suitable source of commercial Letinula edodes inoculum and the degree of colonization was observed over a 15-day to 60-day period as compared to a commercial control.

TABLE 8
Electron beam ionizing radiation treated
mixed sawdust substrate Sterility Check

| Degree and | | Actual | | | |
|---|---|---|---|---|---|
| Number of | Conveyer | Dose of | | | |
| Rotations | Speed | radiation | PDA | TSA | TSB |
| of Sample | (FPM) | (kGy) | Plate | Plate | Plate |
| Rotated by | 4.92 | 10 for each | No | No | Not |
| 90° (4x | | exposure | Contami- | Contami- | deter- |
| exposure) | | | nation | nation | mined |
| Rotated by | 4.92 | 10 for each | No | No | Not |
| 180° (2x | | exposure | Contami- | Contami- | deter- |
| exposure) | | | nation | nation | mined |

In another example, further study was conducted by irradiation of Lentinula edodes (Shiitake) mushroom growing substrates comparing the effect of ionizing radiation using a 10 Megaelectron Volt (MeV) electron beam irradiation and a 7 MV (megavolt) X-ray irradiation to compare the level of bioburden reduction achieved by the two technologies.

Shiitake mushroom growing substrates (hardwood sawdust amended with wheat bran, corn, chalk and gypsum, adjusted to between 60 and 65% moisture content was prepared and a sample (between 250 and 500 grams of substrate) was retained in a plastic bag, tightly closed, and kept at room temperature as a control. Bags of 1.4 kg were then treated with different irradiation treatments to determine the exposure required to achieve a bioburden reduction of 10e6 or better. The results are shown in Table 9. The prepared samples were exposed to doses of 10, 15, 20, 25 kGy (Electron beam, 10 bags per dose) and 10, 15, 20, 25 kGy (X-ray, 9 bags per dose). In addition, a set of ten bags were steam sterilized as an additional control.

TABLE 9
Level of Bioburden Reduction Testing

| | | | Sample | Bioburden expressed as CFU/gram | | | | | | | |
|---|---|---|---|---|---|---|---|---|---|---|---|
| | | | Weight | Initial | | | | | | | |
| Substrate | Treatment | Repetition | (gr) | tube | 10-1 | 10-2 | 10-3 | 10-4 | 10-5 | 10-6 | 10-7 |
| Shiitake | Before | 1 | 1 | >150 | >150 | 61 | 12 | 8 | 2 | 2 | 1 |
| | Sterilization | | | | | | | | | | |
| Shiitake | 10 X-Ray | 1 | 1 | >150 | >150 | 49 | 4 | 0 | 0 | 0 | 0 |
| Shiitake | 15 X-Ray | 1 | 1 | 0 | 0 | 0 | 0 | 0 | 0 | 0 | 0 |
| Shiitake | 20 X-Ray | 1 | 1 | 0 | 0 | 0 | 0 | 0 | 0 | 0 | 0 |
| Shiitake | 25 X-Ray | 1 | 1 | 0 | 0 | 0 | 0 | 0 | 0 | 0 | 0 |
| Shiitake | 10 E-Beam | 1 | 1 | 18 | 2 | 0 | 0 | 0 | 0 | 0 | 0 |
| Shiitake | 15 E-Beam | 1 | 1 | 0 | 1 | 0 | 0 | 0 | 0 | 0 | 0 |
| Shiitake | 20 E-Beam | 1 | 1 | 0 | 0 | 0 | 0 | 0 | 0 | 0 | 0 |
| Shiitake | 25 E-Beam | 1 | 1 | 0 | 0 | 0 | 0 | 0 | 0 | 0 | 0 |
| Shiitake | Steam | 1 | 1 | 0 | 0 | 0 | 0 | 0 | 0 | 0 | 0 |
| | Sterilized | | | | | | | | | | |

Note:
Shiitake indicates Lentinula edodes mushroom growing substrate

Results indicate that X-ray treatment at 10 kGy was not sufficient to reduce the bioburden below the targeted level. At exposure at levels of 15 kGy or higher, X-ray irradiation exposure is an additional form of ionizing radiation that can be used to produce a sterilized substrate for the growth of fungal biomass.

Although the invention has been described in terms of particular embodiments in this application, one of ordinary skill in the art, in light of the teachings herein, can generate additional embodiments and modifications without departing from the spirit of, or exceeding the scope of, the claimed invention. Accordingly, it is understood that the descriptions herein are proffered only to facilitate comprehension of the invention and should not be construed to limit the scope thereof.

What is claimed is:

1. A continuous process for the sterilization of a substrate, the process comprising the steps of:
a. preparing a substrate mixture having a moisture content;
b. loading a carrier with the substrate mixture;
c. exposing the substrate mixture to electron beam or X-ray ionizing radiation in an electron beam or X-ray bunker housing to create a sterilized substrate having the same moisture content as the substrate mixture; and
d. unloading the sterilized substrate from the carrier such that the carrier can be reused in the continuous process.

2. The continuous process of claim 1, further comprising a step of packaging the sterilized substrate into a package for transport.

3. The continuous process of claim 1, further comprising a step of inoculating the sterilized substrate with an inoculum to create an inoculated substrate.

4. The continuous process of claim 3, further comprising a step of packaging the inoculated substrate into a package for transport.

5. The method of claim 4, wherein the inoculum is an organism selected from the group consisting of Agaricus spp., Morchella spp., Trichoderma spp., Beauvaria spp., Lentinula spp., Ganoderma spp., and Metahrizium spp.

6. The continuous process of claim 1, wherein during the step of unloading the sterilized substrate, the carrier is taken to a clean room directly adjacent the electron beam or X-ray bunker housing after being exposed to the electron beam or X-ray ionizing radiation, respectively, and the step of unloading takes place within the clean room.

7. The continuous process of claim 1, wherein during the step of unloading the sterilized substrate, the carrier is transported to a clean room that is not directly adjacent the electron beam or X-ray bunker housing after being exposed to the electron bean or X-ray ionizing radiation, respectively.

8. The continuous process of claim 1, wherein the substrate mixture includes nutrient particles and optionally added water.

9. The continuous process of claim 8, wherein the nutrient particles are selected from the group consisting of sawdust, fermented sawdust, millet, sorghum, grain, oilseed products, agricultural waste, cereal, formulated compost, fermented compost, peat moss, gypsum, chalk, vermiculite, perlite, clay, wastepaper materials, and combinations thereof.

10. The continuous process of claim 1, wherein the moisture content of the substrate mixture is between 30% and 70%.

11. The continuous process of claim 1, wherein the step of exposing includes subjecting the substrate mixture to an ionizing radiation dosage of between about 5 kGy to about 40 kGy.

12. The continuous process of claim 1, wherein the step of exposing takes place at a temperature of from between about 10 °C and about 35 °C.
